(19) United States
(12) Patent Application Publication
(10) Pub. No.: US 2014/0081131 A1
(43) Pub. Date: Mar. 20, 2014

(54) ANGIOGRAPHIC EXAMINATION METHOD

(71) Applicant: Yiannis Kyriakou, Spardorf (DE)

(72) Inventor: Yiannis Kyriakou, Spardorf (DE)

(21) Appl. No.: 14/012,014

(22) Filed: Aug. 28, 2013

(30) Foreign Application Priority Data
Sep. 18, 2012 (DE) 102012216652.1

(51) Int. Cl. A61B 6/00

(52) U.S. Cl. CPC A61B 6/504
USPC 600/425

(57) ABSTRACT

A method is provided for angiographic examination of an organ, vascular system or other body regions as the examination object of a patient by means of 4D rotational angiography. A step S1 of the method involves acquisition of projection images in different cardiac phases. A further step S2 involves reconstruction of 3D volume images in the different cardiac phases. A further step S3 involves calculation of a motion map. A further step S4 includes image combination of the 3D volume images with the motion map to produce resulting, corrected 3D volume images in the different cardiac phases. A further step S5 involves presentation of the resulting, corrected 3D volume images.

### CROSS REFERENCE TO RELATED APPLICATIONS

This application claims priority of German Patent Office application No. 102012216652.1 DE filed Sep. 18, 2012. All of the applications are incorporated by reference herein in their entirety.

### FIELD OF INVENTION

The invention relates to an angiographic examination method for an organ, vascular system or other body regions as the examination object of a patient by means of 4D rotational angiography.

### BACKGROUND OF INVENTION

Such an angiographic examination method as mentioned above can be performed for example with an angiography system as known from U.S. Pat. No. 7,500,784 B2, which is described below with reference to FIG. 1.

Standard 4D rotational angiography results in reconstructions of individual volumes per cardiac phase. These individual volumes are typically influenced to a significant degree by streak artifacts, which result from the small number of available projections per cardiac phase.

4D rotational angiography, a so-called 4D DynaCT®, can be performed with a number of rotations or just one rotation may suffice. With standard methods the number of available projections per phase is significant. With 4D DynaCT® there are generally 30 projections per phase with one rotation. Streak artifacts are therefore present in the reconstructed layers, as described below. The fewer projections are used, the more streak artifacts result in the reconstruction, as this type of reconstruction does not use any redundant information.

Other methods known from the literature operate with iterative reconstruction and minimization methods based on raw data, as described for example in “Prior image constrained compressed sensing (PICCS): A method to accurately reconstruct dynamic CT images from highly undersampled projection data sets” by Guang-Hong Chen et al., published in Med Phys. 2008 February, Vol. 35, No. 2, pages 660 to 663. This is generally very complex and requires a new reconstruction chain.

FIG. 1 shows by way of example an illustrated biplanar x-ray system for performing 4D rotational angiography with two C-arms 2 and 2′ held respectively by a stand 1 and 1′ in the form of a six-axis industrial or buckling arm robot, with an x-ray radiation source, for example x-ray emitters 3 and 3′ with x-ray tubes and collimators, and an x-ray image detector 4 and 4′ as the imaging recording unit positioned respectively at their ends. The stand 1 here is mounted on the floor 5, while the second stand 1′ can be attached to the ceiling 6.

The buckling arm robot known for example from U.S. Pat. No. 7,500,784 B2, which preferably has six rotation axes and therefore six degrees of freedom, can be used to move the C-arms 2 and 2′ as required spatially, for example by rotating them about their centers of rotation between the x-ray emitters 3 and 3′ and the x-ray image detectors 4 and 4′. The inventive angiographic x-ray system 1 to 4 can be rotated in particular about centers of rotation and rotation axes in the C-arm plane of the x-ray image detectors 4 and 4′, preferably about the center point of the x-ray image detectors 4 and 4′ and about rotation axes intersecting the center points of the x-ray image detectors 4 and 4′.

The known buckling arm robot has a base frame, which is mounted in a fixed manner for example on the floor 5 or on the ceiling 6. A carousel is fastened thereto in such a manner that it can be rotated about a first rotation axis. A robot link is attached to the carousel in such a manner that it can be pivoted about a second rotation axis with a robot arm fastened thereto in such a manner that it can be rotated about a third rotation axis. A robot hand is attached to the end of the robot arm in such a manner that it can be rotated about a fourth rotation axis. The robot hand has a fastening element for the C-arm 2 or 2′, which can be pivoted about a fifth rotation axis and can be rotated about a sixth rotation axis running parallel thereto.

The implementation of the x-ray diagnosis facility is not dependent on the industrial robot. Standard C-arm devices can also be used.

The x-ray image detectors 4 and 4′ can be rectangular or square flat semiconductor detectors, which are preferably made of amorphous silicon (a-Si). However integrating and possibly counting CMOS detectors can also be used.

Present in the beam path of the x-ray emitters 3 and 3′ is a table plate 7 of a patient support table 8 for holding a patient to be examined as the examination object. The patient support table 8 is provided with an operating console 9. Connected to the x-ray diagnosis facility is a system control unit 10 with an image system 11, which receives and processes the image signals from the x-ray image detectors 4 and 4′ (operating elements are not shown for example). The x-ray images can then be viewed on display units of a monitor bank 12. The image system 11 has an apparatus, the function of which will be described in more detail.

Instead of the x-ray system shown by way of example in FIG. 1 with the stands 1 and 1′ in the form of the six-axis industrial or buckling arm robot, the angiographic x-ray system can also have a standard ceiling or floor-mounted support for the C-arm 2, as illustrated in simplified form in FIG. 2 of U.S. Pat. No. 7,500,784 B2.

Instead of the C-arms 2 and 2′ shown by way of example, the angiographic x-ray system can also have separate ceiling and/or floor-mounted supports for the x-ray emitters 3 and 3′ and x-ray image detectors 4 and 4′, which are coupled for example in an electronically rigid manner.

A method for automatically determining an optimum cardiac phase for a cardio-CT reconstruction is known from DE 10 2007 029 731 A1, in which the following takes place:

sampling a cardiac region of a patient using spiral CT along a z axis and reconstructing a plurality of tomographic image datasets at different z positions with a first resolution,

measuring cardiac activity, determining the cycles and cycle phases of the heart and assigning them to the reconstructed image datasets with the first resolution,

generating a motion map,

masking the motion map in respect of one cardiac cycle in each instance.

determining two motion minima for each masked region in the motion map and assigning the minima to the systolic or diastolic end phase of the heart,

reconstructing at least one image dataset with measurement data relating to the determined cardiac phase of at least one of the determined minima with a second resolution, and

displaying this at least one reconstructed image dataset with the second resolution.

In “Improvement of CardiaC CT-Reconstruction using local motion vector fields” by Carsten Oliver Schirra et al., Computerized Medical Imaging and Graphics; Vol. 33; pp. 122-130, to reduce motion blur and improve the signal to noise ratio (S/N), a motion-corrected reconstruction is described, which uses local motion vector fields of high-contrast objects for motion correction during filtered backprojection. Image registration is performed during a quiet cardiac phase. Temporal interpolation in the parameter space serves to determine motion during cardiac phases with significant motion. The resulting motion vector fields are used during image reconstruction.

### SUMMARY OF INVENTION

The invention is based on the object of configuring an angiographic examination method of the type mentioned in the introduction so that a reduction of streak artifacts is suppressed in heart-correlated 4D rotational angiography, so-called DynaCT®.

According to the invention the object is achieved for an angiographic examination method of the type mentioned in the introduction by the features cited in independent claim(s). Advantageous configurations are cited in the dependent claims.

According to the invention the object is achieved for an angiographic examination method by the following steps:

acquisition of projection images in different cardiac phases and positions,

reconstruction of 3D volume images in the different cardiac phases from the projection images,

calculation of a motion map from the 3D volume images,

image combination of the 3D volume images with the motion map to produce resulting, corrected 3D volume images in the different cardiac phases and

presentation of the resulting, corrected 3D volume images.

This inventive method utilizes redundant data to reduce the streak artifacts in the heart-correlated 4D rotational angiography images, for example with DynaCT®.

### BRIEF DESCRIPTION OF THE DRAWINGS

The invention is described in more detail below with reference to exemplary embodiments illustrated in the drawing, in which:

FIG. 1 shows a known biplanar C-arm angiography system with an industrial robot as support apparatus in each instance,

FIG. 2 shows the relationships for an EKG correlated acquisition during a rotation with a rotational angiography system according to FIG. 1,

FIG. 3 shows a series of projection images acquired according to a standard rotational angiography method according to FIG. 2,

FIG. 4 shows the production of a motion map from reconstructed 3D volume images,

FIGS. 5 to 8 show diagrams to illustrate the postprocessing of the motion map produced according to FIG. 4,

FIG. 9 shows an illustration of a linear image combination with linear interpolation and

FIGS. 10 to 13 show illustrations of the time sequence of postprocessing and its results.

### DETAILED DESCRIPTION OF INVENTION

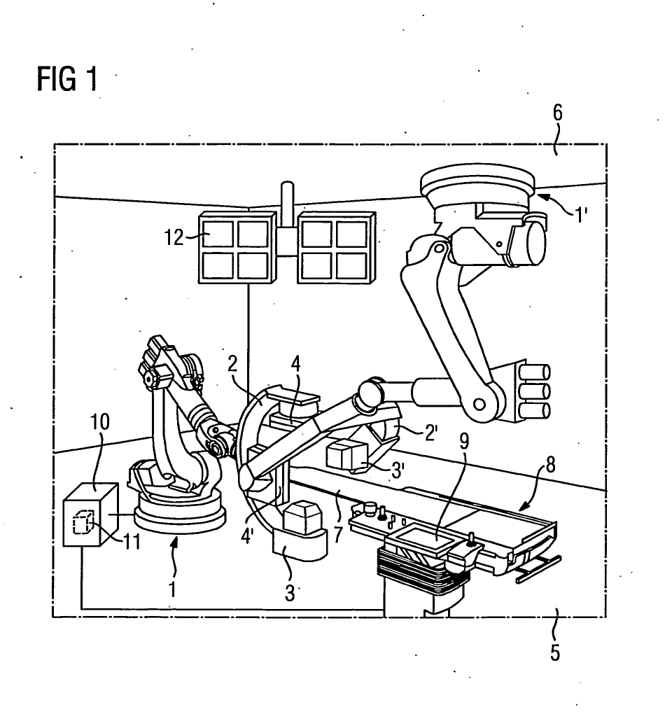

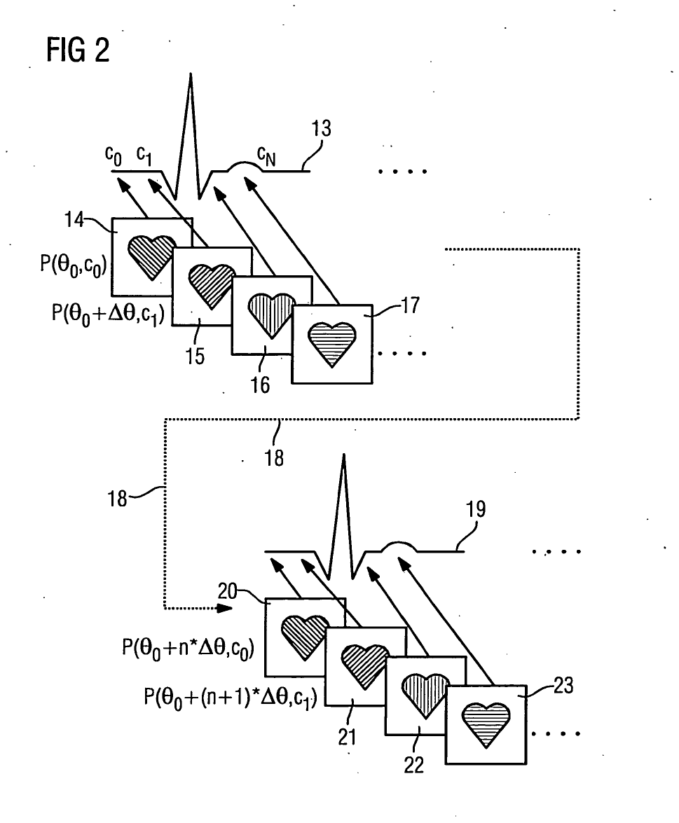

FIG. 2 shows the relationships for EKG-correlated acquisition with a C-arm device according to FIG. 1 during a rotation, as performed at a heart rate of 90 to 131 bpm for a duration of 10 s to 15 s and with or without cardiac phase control (pacing). If pacing does not take place, a known manual sorting of the phases from the EKG is brought about.

This figure shows a first EKG 13, which has different cardiac phases c₀ to c_{N}. Assigned to these cardiac phases c₀ to c_{N} are different projection angles θ0 to θ0+n*Δθ. Thus for a first image 14 of a first cardiac phase c0 a value P(θ₀, c₀) results, for a first image 15 of a second cardiac phase P(θ₀+Δθ, c₁), for a first image 16 of a third cardiac phase P(θ₀+2Δθ, c₂) and for a first image 17 of an Nth cardiac phase P(θ₀+NΔθ, c_{N}) P(θ₀+NΔθ, c_{N}).

This continues as symbolized by the arrow 18 until a second EKG 19 is reached.

Different projection angles θ0+n*Δθ to θ0+(n+N)*Δθ are again assigned to these cardiac phases c₀ to C_{N}. Thus for a second image 20 of a first cardiac phase c₀ a value P(θ₀+nΔθ, c₀) results, for a second image 21 of a second cardiac phase P(θ₀+(n+1)Δθ, c₁), for a second image 22 of a third cardiac phase P(θ₀+(n+2)Δθ, c₂) and for a second image 23 of an Nth cardiac phase P(θ₀+(n+N)Δθ,c_{N}).

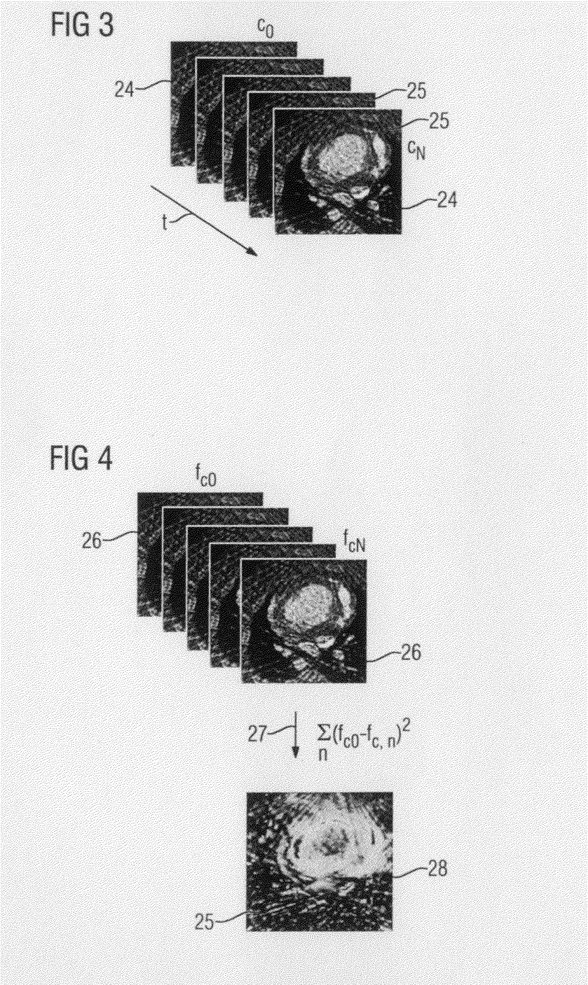

FIG. 3 shows the series of projection images 24 produced according to a standard method with approx. 30 projections per cardiac phase at 120 bpm and 13 s scan time with interfering streak artifacts. The indices c₀ to C_{N} designate the projection images 24 of the current cardiac phases.

FIG. 4 shows a sequence of reconstructed 3D volume images 26, produced with approx. 30 projections per cardiac phase, from which a calculation 27 is performed of an image-based motion map 28 according to the formula

$${\sum }_{n} {( {f}_{c 0} - {f}_{c , n} )}^{2} .$$

The indices f_{c0} to f_{cN} of the 3D volume images 26 designate the reconstructed 3D volume for the corresponding cardiac phase (c₀ to C_{N}) and contain the image information.

As the motion map 28 also features interfering streak artifacts 25, postprocessing is performed on the motion map 28, as described in more detail with reference to FIGS. 5 to 8.

One method is analysis in the frequency domain. In FIG. 5 in a 3D volume image 26 and the motion map 28 two representatively selected pixels 29 and 30 are considered, of which the first pixel 29 features significant motion at low frequency and the second pixel 30 features little motion at high frequency.

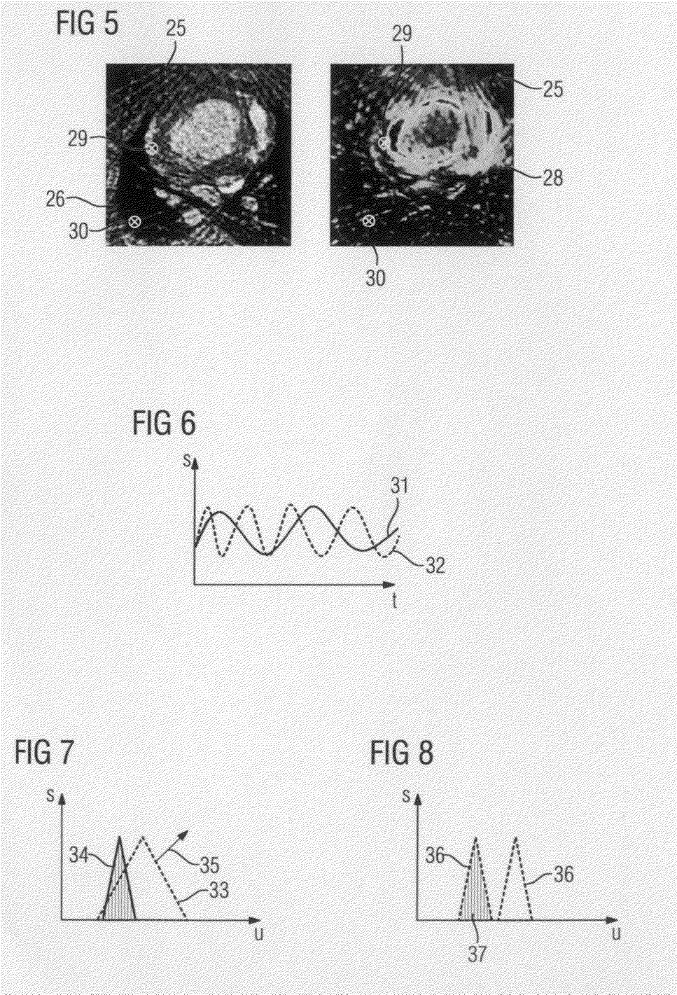

FIG. 6 shows the signal profiles of the pixels 29 and 30, the signal profile 31 of the first pixel 29 having a lower frequency than the signal profile 32 of the second pixel 30.

In FIG. 7 data relating to the modulation of heart motion and streak artifacts 25 is plotted over spatial frequency u, showing a modulated signal profile 33 of the first pixel 29 and a modulated signal profile 34 of the second pixel 30, which have a modulation direction 35.

FIG. 8 shows data after demodulation of heart motion and streak artifacts 25 plotted over spatial frequency u with a demodulated signal profile 36 of the first pixel 29 and a demodulated signal profile 37 of the second pixel 30.

The principle of modulation and demodulation essentially means that at some points, for example at the second pixel 30, the pixel values only change quasi-periodically due to the streak artifacts 25. These quasi-periodic changes of the streak artifacts 25 are based on the so-called windmill effect. They are sampling artifacts as a function of time. At other points, for example at the first pixel 29, the change to said pixel 30 can be traced back as a function of time to the windmill effect and heart motion artifacts. This type of change should be identified to process such selective diffusion with filters, for example demodulation.

The principles of modulation and demodulation are generally known from signal theory or signal processing; Fourier analysis or band filtering can be used here.

Modulation is defined by the recording itself; demodulation is used to isolate the “carrier” signal from the “true” signal. With the type of recording specified here this is relatively simple, as the windmill artifacts have quite a defined frequency, which is only a function of the recording geometry and can therefore be calculated easily beforehand.

Morphological operations such as for example erosion and/or dilatation of the motion map 28 can be used as further methods for postprocessing the motion map 28.

The for example bilinear or spline subsampling and interpolation method can also be used for postprocessing the motion map 28.

As a result of postprocessing the motion map 28 using one of these methods, a corrected motion map is obtained, which is almost free of streak artifacts 25.

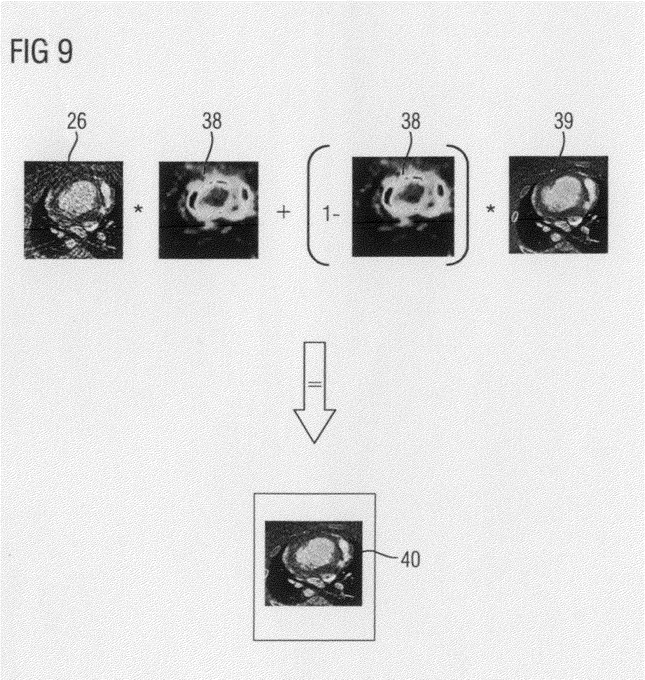

One example of an image combination shown in FIG. 9 is a linear combination with linear interpolation. However other types of combination are also possible, for example polynomial or quadratic image combinations. Image combinations with a convolution operator are also conceivable.

One of the possible image combinations, which results generally from the following equation, is now described with reference to FIG. 9:

F(x, y, z, cₙ)=f(x, y, z, cₙ)*MM(x, y, z)+ f̅(x, y, z)*(1−MM(x, y, z))

where cₙ represents the respective cardiac phase c₀ to c_{N}.

The pixels of the reconstructed 3D volume images 26 f(x, y, z, cₙ) are multiplied by the pixels of the corrected motion map 38 MM(x, y, z). Added to this is the product of one minus corrected motion map 38 MM(x, y, z) and the mean value image 39 f̅(x, y, z) over all phase images. The result F(x, _{y, z, c}ₙ) is the resulting, corrected 3D volume images 40.

This multiplication represents the simplest instance of an image combination, in which a pixel or voxel-based multiplication (weighting) of the two images (or volumes) is always performed per phase, with the motion map remaining constant after postprocessing.

In other words the result for the example of the first cardiac phase c₀ would appear as follows:

Fc₀(x, y, z)=fc₀(x, y, z)*MM(x, y, z)+ f̅(x, y, z)*(1−MM(x, y, z))

This is shown thus by way of example for a linear interpolation. In the case of a non-linear combination a corresponding function f(MM(x,y,z)) would have to be defined, e.g. polynomially. In the present instance it is mainly a matter of weighting the individual volumes according to the motion map.

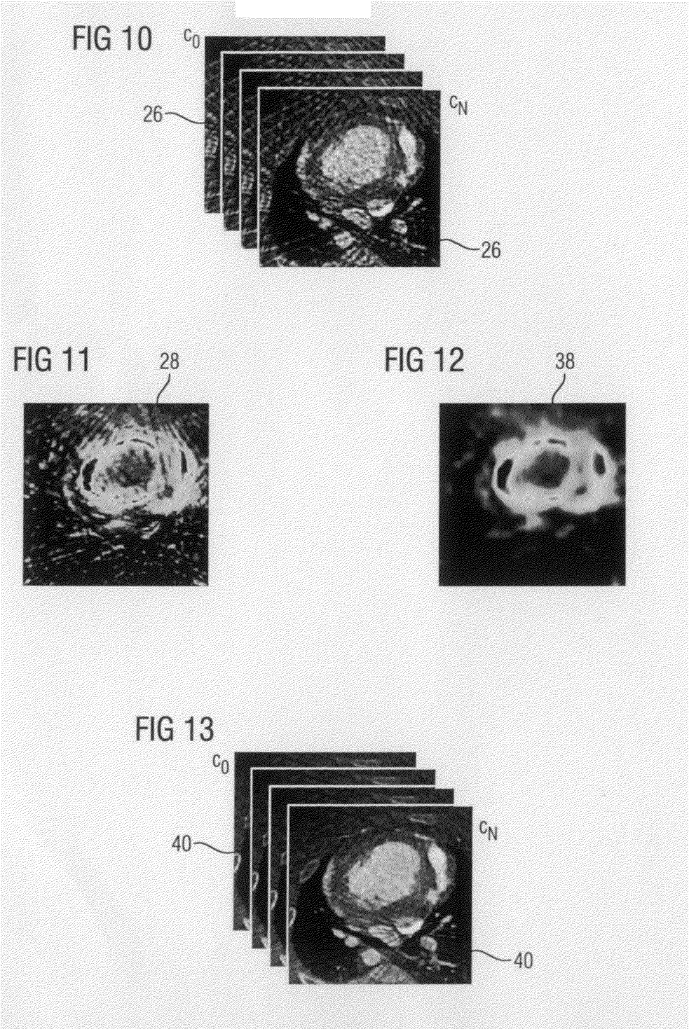

The result of postprocessing can also be described in more detail and illustrated symbolically based on FIGS. 10 to 13, which show the time sequence of image production. The starting point is the image series “before motion map postprocessing” of the reconstructed 3D volume images 26. The motion map 28 is calculated therefrom. This motion map 28 is then corrected based on the processing described in FIGS. 5 to 8 to produce a “motion map postprocessing” of the corrected motion map 38. Finally the resulting, corrected 3D volume images 40 “after motion map postprocessing” are calculated according to the above equation.

The method proposed above operates on the basis of the reconstructed layers, the 3D volume images 26.

One type of acquisition is rotation with effective angle sampling, for example a sampling time of 13 s, 0.5° angle increment and 2×2 binning. This produces around 380 projections over all phases. Available redundant information is utilized as only some of the voxels in the image change. The change to the voxels is calculated by means of the motion map 28 per layer. The motion map 28 shows the content of the motion or the change to the voxel values over time. A voxel has a different motion function, in other words change function or gradient, in the heart, from when it is present in a different body part.

The motion map 28 is also influenced by streak artifacts 25 in the first step. To reduce this, three postprocessing methods arte proposed, to isolate changes due to streak artifacts 25 and changes due to pure heart motion. This results in a reduction of the streak artifacts 25 in the motion map 28.

The motion map 28 is utilized as a combination weighting between the reconstruction of an individual phase (e.g. c0) and the mean value image from all phases. It is assumed here that the voxel values in the motion map 28 with a small value contribute less to heart motion.

The image combination can be produced by linear interpolation but other types of combination are also possible.

The resulting corrected 3D volume images 40 have significantly fewer streak artifacts 25.

The inventive method can be used for monoplanar and biplanar systems. Unlike many other known methods it is a purely image-based method. It does not require raw data, geometry or other information.

The inventive method eliminates streak artifacts 25 from 4D rotational angiography, so-called 4D DynaCT® images, almost completely with limited loss of spatial and temporal resolution.

The generation and postprocessing of the motion map 28 further reduces interfering streak artifacts 25.

The inventive method can also be used for other protocols with changes in the time direction, for example perfusion.

The available reconstruction chain is utilized effectively for the calculations.

1. An angiographic examination method for an organ, vascular system or other body regions as the examination object of a patient by means of 4D rotational angiography, the method comprising the steps of:
S1) acquiring projection images in different cardiac phases (c₀ to c_{N}) and positions,
S2) reconstructing 3D volume images in the different cardiac phases (c₀ to c_{N}) from the projection images,
S3) calculating a motion map from the 3D volume images,
S4) performing image combination of the 3D volume images with the motion map to produce resulting, corrected 3D volume images in the different cardiac phases (c₀ to c_{N}) and
S5) presenting the resulting, corrected 3D volume images.

2. The angiographic examination method as claimed in claim 1, wherein the 3D volume images are used to form a mean value image f̅(x, y, z) over all the cardiac phases, which is included in the image combination according to method step S4.

3. The angiographic examination method as claimed in claim 1, wherein the resulting, corrected 3D volume images are calculated according to the following equation:
F(x, y, z, cₙ)=f(x, y, z, cₙ)*MM(x, y, z)+ f̅(x, y, z)*(1−MM(x, y, z)),
where
cₙ represents the respective cardiac phase c₀ to c_{N},
f(x, y, z, cₙ) represents the reconstructed 3D volume images,
MM(x, y, z) represents a motion map,
f̅(x, y, z) represents a mean value image over all the phase images and
F(x, y, z, cₙ) represents resulting, corrected 3D volume images.

4. The angiographic examination method as claimed in claim 3, wherein the motion map is a postprocessed, corrected motion map.

5. The angiographic examination method as claimed in claim 1, wherein the motion map is calculated as follows according to method step S3):
$${\sum }_{n} {( {f}_{c 0} - {f}_{c , n} )}^{2} ,$$
where the indices f_{c0} to f_{cN} of the 3D volume images designate the reconstructed 3D volumes for the corresponding cardiac phase (c₀ to c_{N}).
